(19) United States
(12) Patent Application Publication
(10) Pub. No.: US 2006/0086045 A1
(43) Pub. Date: Apr. 27, 2006

(54) Gelatinous moisturizing agent delivery system and method

(76) Inventors: Harold Jensen, Santa Rosa, CA (US); Glenn Smith, Santa Rosa, CA (US); Joseph Paternoster, Santa Rosa, CA (US)

Correspondence Address: Dergosits & Noah LLP, Suite 1450, Four Embarcadero Center, San Francisco, CA, 94111, US

(21) Appl. No.: 10/973,507

(22) Filed: Oct. 26, 2004

(51) Int. Cl. A01H 3/00

(52) U.S. Cl. 47/58.1 R; 504/366

(57) ABSTRACT

A delivery system for distributing water to plant tissue, such as the root ball of a living plant. A hollow delivery tube is provided having a receiving end and an insertion end. The delivery tube is at least partially perforated having side walls although other embodiments are contemplated. A gelatinous moisturizing agent is introduced within the delivery tube which acts to release water to the plant tissue in a time release fashion.

### TECHNICAL FIELD

The present invention relates to the distribution of a moisturizing substrate such as that described in U.S. Pat. No. 4,865,640. More specifically, the present invention relates to a system and method for controllably delivering time release water to plant tissue such as the entire vertical root system of a plant.

### BACKGROUND OF THE INVENTION

The problem of maintaining soil moisture content sufficient to sustain a growing plant has long been recognized. The most common method of adding water to soil is by watering using manual or automatic means, such as sprinkler or drip irrigation systems. However, manual watering is extremely time and labor-intensive. In addition, automatic watering systems can be expensive to install and operate. Also, conventional water methods are historically wasteful.

Moisturizing agents have been used to solve problems inherent to manual and automatic watering systems. A moisturizing agent releases moisture into its immediate vicinity. One such moisturizing agent is described in U.S. Pat. No. 4,865,640 (Avera), the disclosure of which is incorporated by reference. The Avera moisturizing agent is a gel-like product that appears dry to the touch and semi-solid in appearance. This moisturizing agent can contain approximately 98% of water bound in solid form. The solid bound water is gradually converted to liquid water when placed in the microbial environment of natural soils. The bacteria and other microorganisms that liquefy the bound water do not migrate into the gel but act on its exposed surface. The liquefaction rate of moisturizing agents such as that taught by Avera, can therefore be controlled by controlling the amount of moisturizing agent surface area that is exposed to the soil.

FIG. 1 is a side view of a delivery system for a moisturizing agent according to the prior art. The moisturizing agent 12 is disposed within a carton 10. The bottom 20 of the carton is removed to expose the moisturizing agent. The carton is then inserted into a hole 22 in soil 16. The moisturizing agent exposed to the soil through the bottom of the carton is liquefied by microbial action to provide water to the root system 18 of plant 14. As the exposed portion of the moisturizing agent is liquefied, the next layer of bound-water gel becomes exposed to the microorganisms and drips out of the bottom of the carton. When the moisturizing agent has been completely liquefied, the empty carton is removed from the soil.

Several problems are associated with the distribution system of FIG. 1. The carton is unsightly, and therefore is not suitable for use with decorative plants. It is not possible to determine whether the moisturizing agent within the carton has been completely liquefied without removing the carton. Furthermore, the carton must be removed and a new carton inserted to provide a new supply of moisturizing agent. Soil can then filter into the hole made by the removed carton. It can therefore be time consuming and difficult to insert another carton into the hole.

FIG. 2 is a side view of a delivery system for a moisturizing agent according to the prior art exemplified by U.S. Pat. No. 6,138,408. The moisturizing agent 32 is contained in casing 30 similar to a sausage casing. The end of the casing is removed to expose the moisturizing agent. The moisturizing agent is inserted through receiving end 26 of delivery tube 34 and the moisturizing agent contacts the soil at the insertion end 28. A cap 24 is then removeably placed over the receiving end 26 to seal the insert within the hollow bore of the delivery tube. With this delivery system, the moisture is only provided to a small portion of the root ball, as shown in FIG. 1. It would therefore be an advantage if the system provided moisture to the entire vertical depth of the root ball.

A “cup” (not shown) that contains a moisturizing agent has also been used to distribute controlled amounts of water to a plant. The cup is uncovered and placed upside-down on the soil surface to expose the moisturizing agent contained in the cup to microbial action. The cup, however, is unsightly. In addition, this distribution system is not practical for use outdoors because the cup is easily tipped over or moved by wind, rain, or hail. Furthermore, insect colonies can develop in the moist shaded area underneath the cup.

### SUMMARY OF THE INVENTION

The present invention is a system and method for controllable delivery of water from a moisturizing agent to plant tissue, including both growing plants and cut plant displays (such as flower arrangements). In one embodiment of the present invention, an insert containing moisturizing agent is opened and the moisturizing agent is then placed through the receiving end of a delivery tube and into the hollow bore thereof. A cap is then removably placed over the receiving end to seal the insert within the hollow bore of the delivery tube, thereby reducing loss of moisture to evaporation and preventing foreign matter or insects from migrating into the tube. The moisturizing agent is easily replaced by removing the cap and replacing the moisturizing agent within the hollow bore of the delivery tube. The delivery tube of the present invention is perforated in part or entirely as needed by the plants of interest. Alternatively, the delivery system could be a saucer containing the gelatinous moisturizing agent on which a potted plant may be placed for bottom watering through a hole in the bottom of the pot.

The insert is preferably made of thin plastic that encloses moisturizing agent in a sausage-shaped chub. However, the delivery tube is readily configured to receive any suitable shape of moisturizing agent. Alternatively, moisturizing agent can be directly injected into the delivery tube, for example, from a tank, bladder or canister by gravity feed or pumped with a gasoline engine or electric powered pump or a manually powered pump using a crank or pedal system or directly from a mixer using similar power sources.

The amount of water provided to a plant, and the length of time over which this water is provided, are determined by the size of the insert and by the moisturizing agent surface area that is exposed to the soil. The liquefaction rate can be further controlled by adding a small amount of food grade preservative to the moisturizing agent to retard the bacterial action as suggested by U.S. Pat. No. 6,138,408.

### BRIEF DESCRIPTION OF THE FIGURES

FIG. 1 is a side view of a delivery system for a gelatinous moisturizing agent according to the prior art, namely, U.S. Pat. No. 4,865,640;

FIG. 2 is a side view of a prior art delivery tube as disclosed in U.S. Pat. No. 6,138,408;

FIG. 3 is a side sectional view of a perforated tube and a cap as well as examples of cone-shaped bottoms and inserts of gelatinous moisturizing agent;

FIG. 4 is a cut away view of three different tube configurations of the present invention;

FIG. 5 is a side view of a horizontal crop tube and riser/fill tubes of the present invention;

FIG. 6 is a bottom, top and perspective view of a flexible tube that could be used as a tree ring or planting bed watering system in accordance with the present invention;

FIG. 7 is a tank with a cone-shaped bottom for delivering gelatinous moisturizing agent to a perforated tube system, either by gravity feed or by pumping in accordance with the present invention;

FIG. 8 is a piece of partially perforated sheet material with flanges on two to four sides that, when rolled, hook together to form a tube of the present invention;

FIG. 9 shows a cone-shaped perforated tube used to apply this invention to a hanging basket;

FIG. 10 shows the cone-shaped tube of FIG. 9 in three different applications;

FIG. 11 is a customized saucer of the present invention with a film covering having printed cutting guides for different sizes of pots; and

FIG. 12 is a further embodiment of this invention whereby a direct injection probe for injecting the gelatinous moisturizing agent directly into soil is shown.

FIG. 13 is a perforated inverted cone, partially or totally perforated. 13a is a solid inverted cone voider for use in creating a pocket in the soil to accept FIG. 13. 13b is a cap to removeably cover FIG. 13.

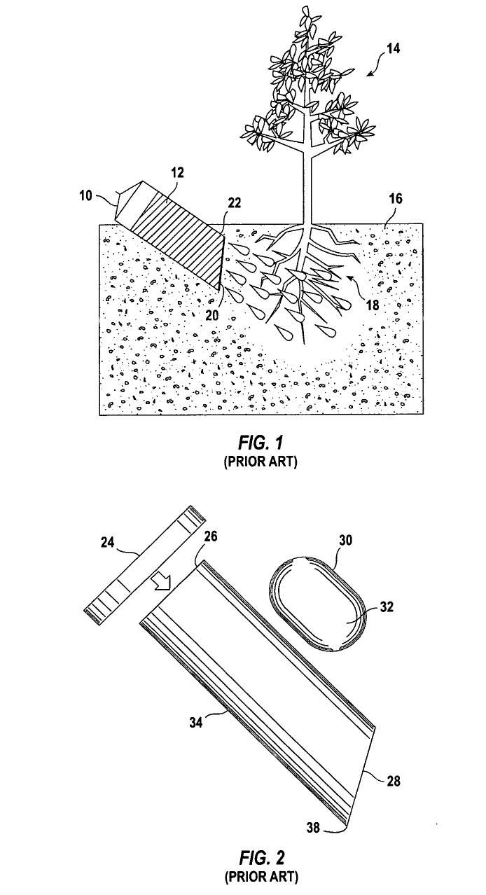

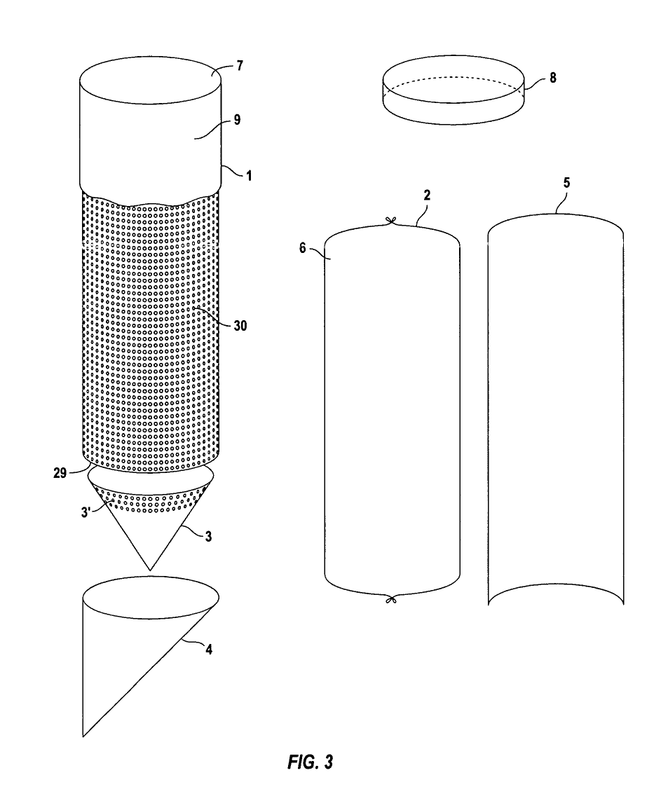

### DETAILED DESCRIPTION OF THE INVENTION

The present invention is a system and method for controllably delivering water from a gelatinous moisturizing agent to plant tissue. The tube delivery system according to the present invention can be used to provide controlled amounts and rates of water distribution to both growing plants and cut plant displays (such as flower arrangements). The preferred embodiment of the present invention is adapted for distributing the moisturizing agent described in Avera, discussed previously. However, the teachings of the present invention can equally be applied to distribution of any other appropriate gelatinous moisturizing agent.

FIG. 3 is a side view of a delivery tube according to the present invention. In the preferred embodiment of the present invention, delivery tube 1 is a hollow length of rigid, perforated tubing made of but not limited to, for example, a non-corrosive material such as plastic, ceramic, paper or other degradable material or glass. However, the delivery tube can also be formed of any suitable material or combination of materials that can support the moisturizing agent insert 2. Such materials include but are not limited to metals, wood, rubber, paper, and natural or artificial fibers. The delivery tube can be formed by such methods as blow molding, injection molding, extruding or fabrication.

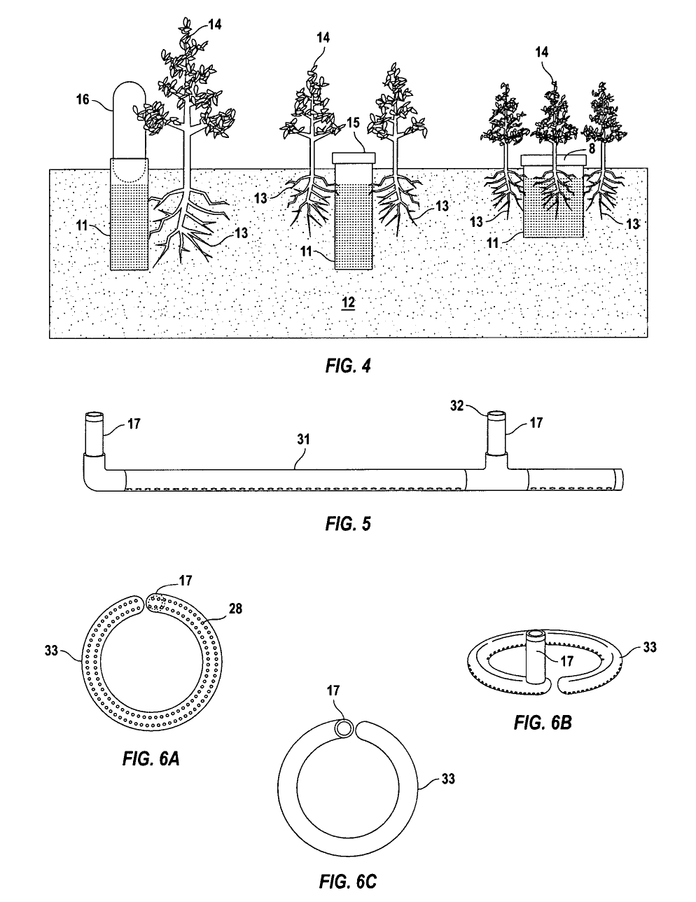

The insertion end 29 of delivery tube 1 can be flat or pointed extensions 3 or 4 frictionally fit thereon to facilitate insertion of tube 1 into soil. The extensions can be of any geometric shape and be provided with perforations 3 or have non-perforated side walls as in the case of extension 4. The delivery tube 1 is placed in soil or similar growth media in close proximity to the root system of a plant (FIG. 4). In the preferred embodiment of the invention, delivery tube 1 is inserted deeply into the soil such that, at most, only a small portion of the length of the delivery tube protrudes therefrom at receiving end 7. However, in alternative embodiments, up to 2 inches of the length of delivery tube 1 can protrude from the soil, depending upon the delivery tube, the depth of the plant root system, and the volume of moisturizing agent to be distributed.

In use, insert 2 containing gelatinous moisturizing agent 6 is opened, the gelatinous moisturizing agent is removed then placed through receiving end 7 and into the hollow bore of delivery tube 1. The extent of perforated area 30 and the size of the perforations will determine the surface area of gelatinous moisturizing agent that is exposed to the microbial action of the soil. A cap 8 is then removably placed over receiving end 7 to seal gelatinous moisturizing agent 6 within the hollow bore of delivery tube 1. The cap can be any depth from 0.5″ to 3″, but not limited to these dimensions. The gelatinous moisturizing agent is easily replaced by removing cap 8, and replacing the gelatinous moisturizing agent within the hollow bore of delivery tube 1. As such, there is no need to remove the delivery tube from the soil to replace the gelatinous moisturizing agent. The cap 8 may be solid to cover some of the perforations in the tube or it can be partially perforated.

As noted previously, the tube delivery system according to the present invention may be provided with perforations 30 around the entire circumference of the delivery tube. To restrict the open area and prevent evaporation through open perforations 30 at or near receiving end 7, a collar 9 of vinyl, rubber, PVC or any other suitable material may be used to cover the perforation 30, for example, on the top three inches of the perforated tube. A solid insert 5 may be used to block moisture release in areas away from the root system. In a preferred embodiment of the present invention, the insert is made of thin plastic tubing that encloses the gelatinous moisturizing agent. This thin tubing can be provided in a variety of lengths and diameters for specific applications. Because the liquefaction rate of the gelatinous moisturizing agent is controlled by the amount of gelatinous moisturizing agent exposed to the soil, the amount of water provided to a plant and the length of time over which this water is provided is dependent upon the size and diameter of the perforated tube.

The hollow bore of the delivery tube is configured to receive a predetermined amount of moisturizing agent. In the presently preferred embodiment, insert 2 is shaped as a chub, resembling a sausage. An exemplary embodiment of the invention is a delivery tube having a length of 4 to 24 inches and a diameter of 1 to 12 inches. The insert can also be formed of other suitable materials or combinations of materials including but not limited to paper, metal, metal foil, and rubber. As noted from a discussion of other embodiments, the gelatinous moisturizing agent can be directly introduced to a suitable delivery tube without use of insert 2.

In alternative embodiments of the present invention, the insert and delivery tube can have any suitable shapes and dimensions. For example, the delivery tube can be configured to receive the gelatinous moisturizing agent from a carton shaped container. Thus, the present invention can be used with the gelatinous moisturizing agent from a prior art carton. Furthermore, using the tube delivery system according to the present invention, the tube can be easily inspected to determine if its contents have been completely liquefied. In doing so, the gelatinous moisturizing agent can be replaced with minimal effort. A delivery tube can also be configured to receive the gelatinous moisturizing agent from a delivery cup known in the prior art.

As noted above, another embodiment of the present invention, moisturizing agent can be directly injected into the delivery tube. For example, the delivery tube can be manually or automatically refilled with gelatinous moisturizing agent that is stored in a tank, bladder, canister or directly from a mixer. This embodiment is used to advantage for outdoor applications, such as reforestation and agriculture where access is difficult and time consuming. Delivery tubes according to the present invention can be placed in the soil at the time of planting or thereafter. The delivery tubes can then be filled and refilled by directly injecting gelatinous moisturizing agent from a storage tank, bladder, canister or directly from a mixer into the hollow bore of the delivery tube. In areas that are not readily accessible to fixed or mobile storage tanks, inserts such as chub 2 containing gelatinous moisturizing agent can be used to controllably provide water to the plants.

FIG. 4 is a side sectional view of a tube delivery system according to the present invention. The delivery tube 11 is placed into the soil with the perforations in close proximity to root system 13 of plant 14. It is noted that perforations 30 are configured within delivery tube 11 only along a portion of its longitudinal axis proximate to root system 13. The gelatinous moisturizing agent 16 is inserted into the receiving end 15 of the hollow bore of delivery tube 11, exposing the moisturizing agent to the microbial activity in the soil. The cap 8 is then placed on the receiving end 15 to seal the gelatinous moisturizing agent within the hollow bore of the delivery tube. In this preferred embodiment, the cap reduces loss of moisture to evaporation and prevents foreign matter or insects from migrating into the tube.

FIG. 4 further shows placement of insert 16 in tube 11 according to the present invention. In this embodiment, the casing has been removed from gel 16, as the gel is being inserted into the delivery tube 11. The cap can then be placed on the receiving end 15 to seal the insert within the hollow bore of the delivery tube.

In all disclosed embodiments, it is noted that the rate of liquefaction of the gelatinous moisturizing agent can be further controlled by adding a small amount of food grade preservative to the gelatinous moisturizing agent. This preservative can retard the bacterial action and extend the liquefaction period. An example of a preferred preservative is sodium benzoate in the amount of 0.005% to 0.05% of the volume of water by weight as taught in U.S. Pat. No. 6,138,408, the disclosure of which is incorporated by reference.

The delivery tube or cap can be decorated or colored to enhance the appearance for use with decorative plants or flower displays. Additionally, the delivery tube or cap can be shaped to be less visible, for example, by being configured or colored to resemble natural items such as stems, tree stumps, or rock formations. For example, in the preferred embodiment of the present invention, the delivery tube and cap are colored brown to blend in with the soil coloring. However, in alternative embodiments, the delivery tube and cap can have any desired color or colors.

Turning to FIG. 5, a cross sectional view of a horizontal, perforated delivery tube 31 with riser/fill tubes 17 with caps 32 to prevent evaporation of moisture from the gelatinous moisturizing agent is shown. The horizontal delivery tube 31 can be rigid for row crops or flexible. It can act in place of a drip irrigation line. To demonstrate the flexibility of such an embodiment, FIG. 6 shows delivery tube 33 bent into a ring for trees and other configurations for irregular planting areas. The horizontal delivery tubes can be placed 1-3″ below the surface for bedding plants or at greater depths as the plants of interest may require. The horizontal delivery tubes can be filled and refilled with a gelatinous moisturizing agent automatically from a tank, bladder, canister or directly from a mixer by gravity feed or by pumping through riser/fill tubes 17 which can be spaced, for example, two to ten feet apart in a normal installation. As noted, FIG. 6A shows the delivery tube 33 from its bottom surface, while FIGS. 6B and 6C show side perspective and top views respectively. It is noted that perforations 28 are generally restricted to the lower half of delivery tubes 31 & 33.

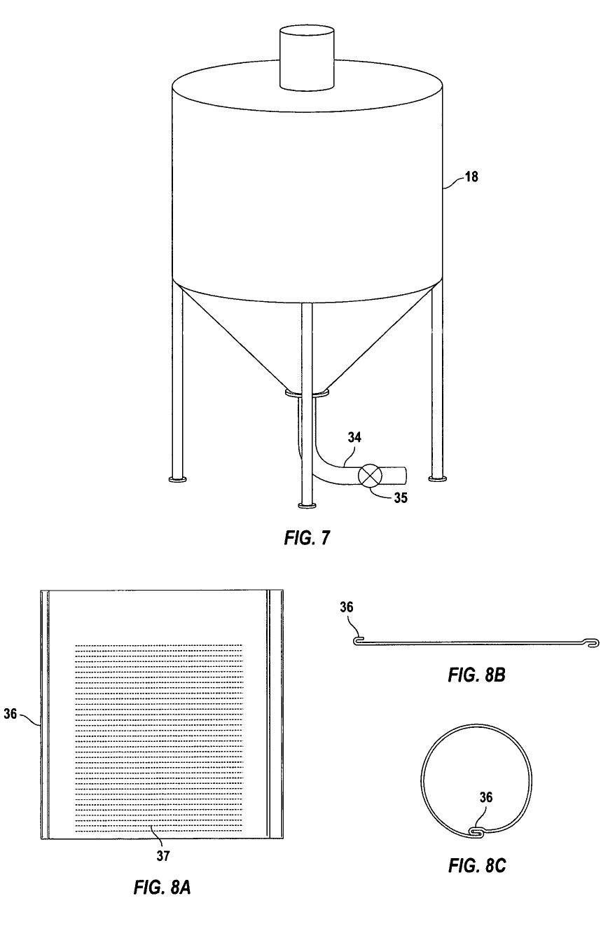

FIG. 7 is an example of a tank 18 that could be used for feeding gelatinous moisturizing agent into perforated tubes by gravity or by pumping through feed line 34 and valve 35.

FIG. 8 shows a flat piece of thin, flexible material such as, but not limited to, for example, plastic, metal, rubber or paper 36 that is perforated partially or wholly with perforations 37 that can be rolled one way to form one size tube or the opposite way to form a different size tube. For example, flexible material 36 could be configured into a 2″ tube 9.5″ long or a 3″ tube 6.5″ long. A cap would be removably placed on one end. The tube would be placed in the soil in close proximity to the root ball of a plant usually in a vertical orientation. The cap would be removed and the gelatinous moisturizing agent would be placed in the tube and the cap replaced. This embodiment as a flat sheet would be of great advantage in warehousing and shipping. FIG. 8A shows the flexible sheet material in plan view while FIGS. 8B and 8C show it in flat and rolled side views respectively.

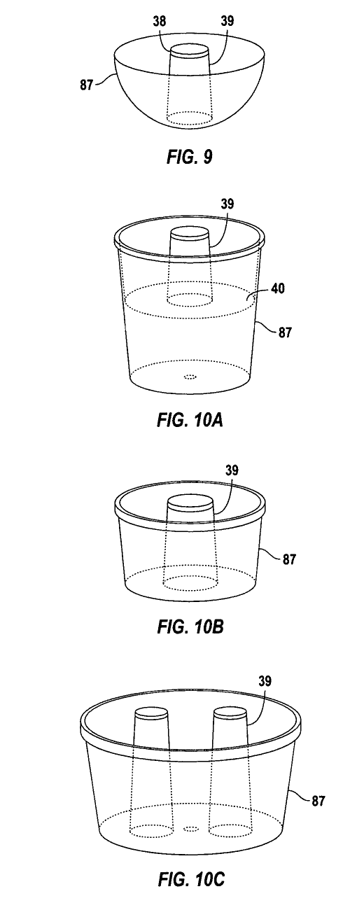

FIGS. 9 and 10 illustrate how cone-shaped perforated tubes could be used in, for example, hanging baskets, potted plants, flower beds and seed beds. There would also be a great advantage in warehousing and shipping of this design as the cone-shaped tubes 39 could be nested until use. A cap 38 could be removably placed on the small end of cone-shaped tube 39. The caps would also be nestable. The tube would be placed in soil (not shown) contained within pot 87. The cap would be removed and the gelatinous moisturizing agent would be placed in tube 39. The cap 38 would then be replaced.

Cone-shaped perforated tube 39 is further shown in FIGS. 9 & 10. Specifically, FIG. 10A shows tube 39 residing upon disk 40 which positions tube 39 in the top half of pot 87. As such, the moisture delivery system of the present invention can be purposely directed to a predetermined location within the pot. By contrast, tube 39 of FIG. 10B is shown extending to the base of pot 87. When dealing with pots of increase volume or when feeding plants having higher water needs, multiple tubes 39 can be employed as depicted in FIG. 10C.

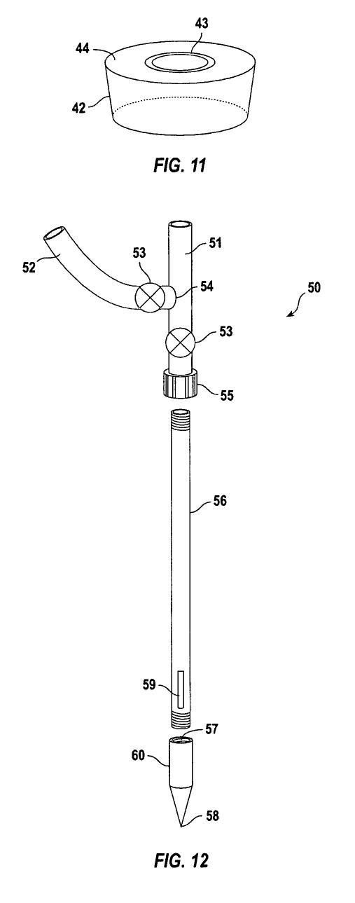

FIG. 11 is directed to saucer 42 with a film 44 covering with a printed cutting guide 43 for different size pots having holes in their bases. Each saucer size could accommodate multiple pot sizes. Some plants prefer bottom watering. The gelatinous moisturizing agent would be placed in the saucer and then sealed with printed film 44 for warehousing and shipping. The user would cut the film for the proper pot size, whereupon the pot is introduced through the hole in the film and onto the gelatinous moisturizing agent.

FIG. 12 is a direct injection probe assembly 50 for directly injecting a gelatinous moisturizing agent into potted soil or in an outdoor application. The probe would include two valves 53, tee joint 54 and screw connector 55, a feed line 51 and pressure and bypass line 52 to enable moisturizing agent to be recirculated when not being injected into the soil. This method of using gelatinous moisturizing agent would be used when faster liquefaction is required to provide more moisture in a shorter period of time without losing moisture to percolation or evaporation. To facility insertion into soil, each member 60 having threaded portion 57 and spike portion 58 can be used. Moisturizing agent would be pumped from a mixer, tank, or other vessel through feed line 51 to probe 56 or bypass line 52. Gelatinous moisturizing agent pumped through probe 56 is dispensed either through perforations (not shown) or through injection port 59.

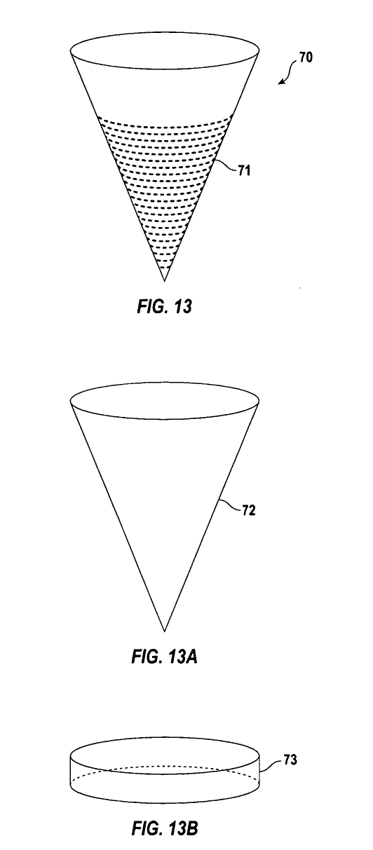

FIG. 13 is a side sectional view of a perforated inverted cone delivery system. This delivery system may be partially or totally perforated. A pocket is formed in the soil, using the solid inverted cone voider 72 of FIG. 13a, to accept the inverted cone delivery system. The inverted cone delivery system consisting of cone 70 having perforations 71 of FIG. 13 is filled with gelatinous moisturizing agent and the cap 73 of FIG. 13b is removeably placed over the inverted cone delivery system to prevent loss of moisture to evaporation. Using the voider 72 to create a pocket for the inverted cone delivery system 70 eliminates the need to dig and avoids cutting roots in the process.

1. A delivery system for distributing water to plant tissue comprising a hollow delivery tube having a receiving end and an insertion end, said delivery tube having an at least partially perforated cylindrically shaped side walls and a gelatinous moisturizing agent contained therein wherein said gelatinous moisturizing agent and perforated delivery tube act to release said gelatinous moisturizing agent in a time release fashion to said plant tissue.

2. The delivery system of claim 1 wherein a cap is provided for selectively covering said receiving end to enable said gelatinous moisturizing agent to be introduced to said hollow delivery tube.

3. The delivery system of claim 1 further comprising an insertion cap for fitting upon the insertion end of said hollow delivery tube.

4. The delivery system of claim 1 wherein said insertion cap is conically shaped to facilitate insertion of said hollow delivery tube to an area surrounding said plant tissue.

5. The delivery system of claim 4 wherein at least a portion of said insertion cap is perforated.

6. The delivery system of claim 1 wherein said cylindrically shaped side walls are entirely perforated.

7. The delivery system of claim 1 wherein a segment of said cylindrically shaped side walls proximate said receiving end is devoid of perforations.

8. The delivery system of claim 1 wherein only a portion of said cylindrically shaped side walls are perforated.

9. The delivery system of claim 8 wherein perforations are configured into said cylindrically shaped side walls in only a portion thereof along its longitudinal axis.

10. The delivery system of claim 9 wherein said perforations are configured into said cylindrically shaped side walls to be proximate to said plant tissue when said delivery tube is positioned to provide said plant tissue with water.

11. The delivery system of claim 1 wherein said plant tissue comprises plant roots.

12. The delivery system of claim 11 wherein said plant roots are imbedded within soil.

13. The delivery system of claim 12 wherein said soil displays biological activity that acts to release water from said gelatinous moisturizing agent.

14. A delivery system for distributing water to plant tissue comprising a hollow delivery tube having a receiving end and an insertion end, said delivery tube having at least partially perforated cup-shaped side walls, a gelatinous moisturizing agent contained within said delivery tube wherein said gelatinous moisturizing agent and perforated delivery tube act to release said gelatinous moisturizing agent in a time release fashion to said plant tissue.

15. The delivery system of claim 1 wherein said gelatinous moisturizing agent is encapsulated with a casing prior to insertion of said gelatinous moisturizing agent within said delivery tube.

16. The delivery system of claim 15 wherein said casing is ruptured for introducing said gelatinous moisturizing agent within said delivery tube.

17. The delivery system of claim 1 wherein said delivery tube comprises flexible side walls configurable to a variety of geometric shapes.

18. The delivery system of claim 17 wherein said receiving end and dispensing ends are connectible for configuring said delivery tube into the shape of a hollow ring.

19. A delivery system for distributing water to plant tissue comprising a hollow delivery tube configured in the shape of a ring and a down pipe emanating from said hollow delivery tube for introducing a gelatinous moisturizing agent thereon, said delivery tube having at least partially perforated side walls for the time release of said gelatinous moisturizing agent proximate said plant tissue.

20. The delivery system of claim 19 wherein said plant tissue comprises the root structure of a living plant surrounded by soil.

21. The delivery system of claim 19 wherein said hollow delivery tube is embedded approximately 2 to 4 inches below the surface of the soil.

22. A delivery system for distributing water to plant tissue comprising a hollow delivery tube having a longitudinal axis oriented substantially parallel to a surface in which it is imbedded, said hollow delivery tube having closed ends and at least one down pipe emanating from said hollow delivery tube for introducing a gelatinous moisturizing agent therein, said delivery tube having at least partially perforated side walls for the time release of said gelatinous moisturizing agent proximate said plant tissue.

23. The delivery system of claim 18 wherein said hollow ring is sized and positioned for the time release of said gelatinous moisturizing agent proximate the root structure of a tree.

24. The delivery system of claim 22 wherein said hollow delivery tube is positioned proximate a drip line.

25. The delivery system of claim 19 wherein said down pipe is provided with a cap for the selective introduction of gelatinous moisturizing agent therein.

26. The delivery system of claim 22 wherein said down pipe is provided with a cap for the selective introduction of gelatinous moisturizing agent therein.

27. A delivery system for distributing water to plant tissue comprising a substantially rectangular, at least partially perforated planar sheet material having at least two parallel formed edges for releasably connecting to one another to create a tube of said planar sheet material, a source of gelatinous moisturizing agent contained within said tube which is releasable through said perforated planar sheet material in a time released fashion to said plant tissue.

28. A delivery system for distributing water to plant tissue comprising a cone-shaped hollow delivery tube having a receiving end and an insertion end, said cone-shaped delivery tube having an at least partially perforated cone-shaped side walls, a gelatinous moisturizing agent contained within said cone-shaped delivery tube which is releasable through said perforated cone-shaped side walls in a time released fashion to said plant tissue and a removable cap for selectively covering said receiving end of said cone-shaped delivery tube.

29. The delivery system of claim 28 wherein said cone-shaped hollow delivery tube is open at its insertion end providing for direct contact between said gelatinous moisturizing agent and the area external to said cone-shaped delivery tube.

30. The delivery system of claim 28 wherein said cone-shaped hollow delivery tube is embedded in soil contained within a planter proximate to the root structure of a plant contained therein.

31. A delivery system for distributing water to plant tissue comprising in combination, a saucer and a pot, said pot being sized to fit within the saucer, said saucer having an internal volume for accepting a quantity of a gelatinous moisturizing agent and a sheet of a film sealing said gelatinous moisturizing agent within said saucer, said sheet of film being printed with at least one circular imprint to enable a user to cut the sheet of film along the circular imprint for creation of an opening therein of a size to accept said pot therethrough thus exposing a bottom surface of said pot to said gelatinous moisturizing agent.

32. The delivery system of claim 31, wherein said bottom surface of said pot is characterized as having at least one opening for the migration of said gelatinous moisturizing agent therethrough.

33. A delivery system for injecting a gelatinous moisturizing agent in soil proximate a root ball of a tree or plant, said delivery system comprises a tubular probe having a first end for receiving said gelatinous moisturizing agent and an insertion end for penetrating said soil to position said insertion end proximate said root ball and a dispensing port located proximate said insertion end for dispensing said gelatinous moisturizing agent.

34. The delivery system of claim 33 wherein said system for injecting gelatinous moisturizing agent further comprises at least one valve for selectively introducing said gelatinous moisturizing agent under pressure to said tubular probe.

35. The delivery system of claim 33 wherein said insertion end is characterized as having a pointed outer surface for facilitating insertion of said tubular probe within said soil.

36. A delivery system for distributing water to plant tissue comprising a hollow delivery tube having a receiving end and an insertion end, said delivery tube having at least partially perforated cylindrically shaped side walls, a gelatinous moisturizing agent contained within an insert sized to fit within said delivery tube wherein said insert and perforated delivery tube act to release said gelatinous moisturizing agent in a time release fashion to said plant tissue.

37. The delivery system of claim 1 wherein said gelatinous moisturizing agent further comprises a preservative.

38. The delivery system of claim 1 wherein said preservative comprises sodium benzoate.

39. The delivery system of claim 28 wherein multiple delivery tubes are nestable to facilitate shipment and storage.

40. The delivery system of claim 36 wherein said insert is cylindrically shaped.
